(19) United States
(12) Patent Application Publication
(10) Pub. No.: US 2022/0031425 A1
(43) Pub. Date: Feb. 3, 2022

(54) DENTAL IMPLANT DRILL AND METHOD OF USING

(71) Applicants: Guy Gunacar, Cincinnati, OH (US); Ismail Gunacar, Cincinnati, OH (US)

(72) Inventors: Guy Gunacar, Cincinnati, OH (US); Ismail Gunacar, Cincinnati, OH (US)

(21) Appl. No.: 17/392,474

(22) Filed: Aug. 3, 2021

Related U.S. Application Data
(60) Provisional application No. 63/060,265, filed on Aug. 3, 2020.

(51) Int. Cl. A61C 1/08

(52) U.S. Cl. CPC A61C 1/084

(57) ABSTRACT

A dental implant drill bit is internally irrigated and guided by markings. The dental implant drill bit also uses a collar to stop the drill. Any size of implant hole required for placing an implant directly corresponds with one size of implant drill bit, thus allowing a single drill bit osteotomy. Using the internally irrigated dental implant drill reduces the time and number of drill bits during osteotomy.

### CROSS REFERENCE TO RELATED APPLICATION

This application claims priority to U.S. Provisional Patent Application entitled “DENTAL IMPLANT DRILL AND METHOD OF USING,” Ser. No. 63/060,265, filed Aug. 3, 2020, the disclosure of which is hereby incorporated entirely herein by reference.

### BACKGROUND OF THE INVENTION

### Technical Field

This invention relates to a dental implant drill and method of using a dental implant drill.

### State of the Art

Dental implants are commonly used for artificial replacements of teeth roots. Dental implant drills are used to prepare dental implant sites. Drilling into the bone is generally referred to as a dental osteotomy procedure. After the osteotomy procedure is completed, with a hole drilled to the desired diameter and depth, the implant body is placed into the hole in the bone. The dental implant drill bits must be sharp, precise and non-injurious to the bone.

The bone/drill interface can become hot during drilling. External irrigation is used to deliver coolant to the drill site. General guidance recommends osteotomies to be completed slowly in order to keep the bone cool, reduce friction while cutting, and reduce the chance of the bone burning, which can cause necrosis (tissue death). Further, osteotomies require a sequential usage of drill bits according to a certain algorithm in order to make the osteotomy opening or hole the appropriate size before placing the implant. However, an osteotomy hole should not be to wide or to invasive. An osteotomy that is to drawn out or using to many drill bits in and out of the osteotomy hole will cause more bone loss and even unintentional over widening of the osteotomy hole, which can cause a loose implant placement, reducing the procedure's success rate.

Using the minimum number of drill bits with minimum drill time minimizes invasiveness and creates a precise and tight osteotomy hole that greatly improves the initial stability and overall success of the implant. In order to minimize bone loss and keep initial stability when the implant is placed, dentists are forced to balance between using as few drills and drill bits as possible to keep the osteotomy tight and the implant site with maximal bone, but slow and irrigated enough in order to make sure the bone does not burn and necrose. Changing drill bits helps in having a new cool drill bit and allowing time for the bone to cool down while changing drill bits.

Accordingly, what is needed is an implant placement system that will minimize the amount of drill bits used in order to maintain maximum stability but will not burn the bone and cause necrosis or other bad side effects.

### DISCLOSURE OF THE INVENTION

The disclosed invention relates to a dental implant drill for making a drilled implant hole for a dental implant.

Disclosed is a dental implant drill bit, the dental implant drill bit comprising: a longitudinal body portion having guide markings; a central bore longitudinally extending through the longitudinal body portion; a proximal end portion having a drill tip; a flange portion disposed in a medial portion of the longitudinal body portion; and a connecting portion having a distal end portion, wherein the distal end portion is configured to couple to a driver. The central bore may be configured to provide irrigation. The drill bit may be disposable.

Disclosed is a dental implant drill, the dental implant drill comprising: a drill bit, the drill bit comprising a longitudinal body portion having guide markings, a central bore longitudinally extending through the longitudinal body portion, a proximal end portion having a drill tip; a flange portion disposed in a medial portion of the longitudinal body portion; and a connecting portion having a distal end portion; and a driver, wherein the distal end portion is configured to couple to the driver. The central bore may be configured to provide irrigation. The drill bit may be disposable.

Disclosed is a method of using a dental implant drill for a dental osteotomy comprising: drilling a hole using the dental implant drill, wherein said hole is of a desired width and depth for receiving an implant, and wherein the dental implant drill comprises a drill bit, the drill bit comprising a longitudinal body portion having guide markings, a central bore longitudinally extending through the longitudinal body portion, a proximal end portion having a drill tip, a flange portion disposed in a medial portion of the longitudinal body portion, and a connecting portion having a distal end portion, wherein the distal end portion is configured to couple to a driver; and internally irrigating the drill bit through the central bore while drilling said hole. The drill bit for drilling the hole for receiving the implant may be singular. The drill bit may be disposable.

Disclosed is a dental implant drill bit kit comprising one or more drill bits, wherein each of said one or more drill bits comprises: a longitudinal body portion having guide markings; a central bore longitudinally extending through the longitudinal body portion; a proximal end portion having a drill tip; a flange portion disposed in a medial portion of the longitudinal body portion; and a connecting portion having a distal end portion, wherein the distal end portion is configured to couple to a driver. The central bore may be configured to provide irrigation. The one or more drill bits may be of different widths. The flange portion of each of the one or more drill bits may be of a different depth. The one or more drill bits may be disposable.

The foregoing and other features and advantages of the invention will be apparent to those of ordinary skill in the art from the following more particular description of the invention and the accompanying drawings.

### BRIEF DESCRIPTION OF THE DRAWINGS

FIG. 1 shows a plane view of a dental implant drill bit according to the invention.

### DETAILED DESCRIPTION OF EMBODIMENTS OF THE INVENTION

As discussed above, embodiments of the present invention relate to a dental implant drill and method of use. The dental implant drill is internally irrigated and guided by markings, such as stripes, and having a flange or collar in order for the dental implant doctor to guide and stop the implant drill.

Only one drill is used to complete the drilling before placing the implant. Drilling the full and correct dimension of the hole for the implant is performed with a singular drill and singular drill bit without the use of any pilot drills or expanding drills resulting in a one-drill osteotomy.

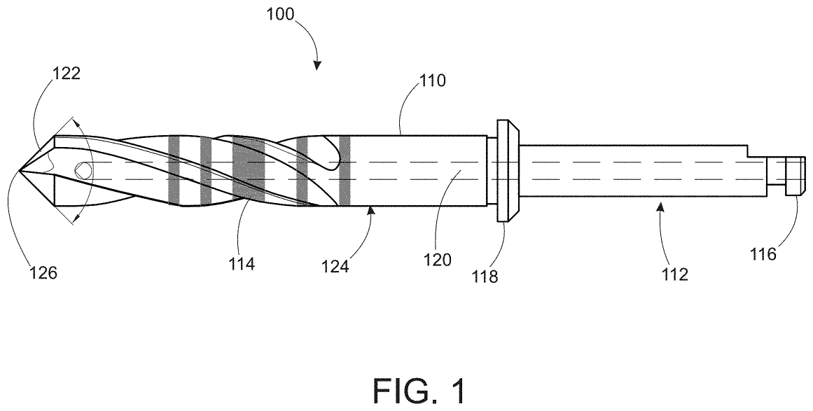

FIG. 1 shows a drill bit 100 having a longitudinal body portion 110. Longitudinal body portion 110 includes a proximal end 122 and a distal end 116. A central bore extends longitudinally through the longitudinal body portion 110 from the proximal end 122 to the distal end 116. A connecting portion 112 of longitudinal body portion 110 extends from distal end 116 to a flange 118. A medial portion 122 of longitudinal body portion 110 extends from flange 118 to proximal end 122. Connecting portion 112 connects at distal end 116 to a driver or chuck (not shown). An indexing or guide feature 114 is located on longitudinal body portion 110. Proximal end 122 is provided with a drill tip 126 and aligned with a vertical axis of longitudinal body portion 110. Central bore 120 longitudinally extending through longitudinal body portion 110 provides irrigation. The internally irrigated and guided drill prevents bone from burning by cooling the bone.

A flange or collar portion 118 is disposed in a medial portion 124 of longitudinal body portion 110. In another embodiment, flange 118 is shifted on longitudinal body portion 110 towards proximal end 122. Different sized drills, with varyingly spaced flanges are used for creating implant placement holes at various required depths for proper implant placement. The flange is spaced further down for implant holes that need to be drilled deeper. When drilling, flange or collar 118 comes into contact with the surgical guide (usually placed on top of the surgical site), thus stopping it from going to deep when drilling. Flange or collar 118 stops or prevents the dental drilling from going to deep into the bone of a patient.

Generally, indexing or guide markings 114 are used sequentially beginning with a 7.0, 8.5, 10.0, 11.5, 13.0 and 15.0 mm as measured from proximal end 122. Other markings may be used.

Indexing or guide markings 114 on dental implant drill bit 100 allow a single drill to be precise without using sequential drills. Whereas other drill bits or drill bit systems use multiple and sequential drill bits and drills make a single implant hole, the design of this implant allows for each depth and size of implant drill hole to correspond with one and only implant drill bit, thus saving time, minimizing bone loss, and reducing invasiveness of the procedure

Some embodiments comprise a kit of drill bits having different widths and having collars of different depths. The kit may comprise at least two drill bits, generally three or more drill bits of different widths and having collars of different depths. The internally irrigated and guided drill bits may be disposable to maintain sharpness.

Dental implant drill bit 100 is used for drilling a dental implant hole for a dental implant. The method of using the dental implant drill according to the invention comprises selecting the specific internally irrigated and guided drill bit that corresponds to the size and depth of the osteotomy hole needed to place a planned implant. This is in contrast to the conventional method of using a sequential set of drill bits in an algorithm, each one slowly widening the osteotomy hole until the osteotomy is the appropriate size for placement of the planned implant.

The method of using the dental implant drill according to the invention reduces the time for a surgery (osteotomy procedure) for one dental implant from about 30-40 minutes to a time in the range of from about 5 to about 10 minutes, generally about 5 minutes. When doing a surgery (osteotomy procedure) of over 30 minutes, the procedure is slower and less heat is generated making irrigation less necessary. In addition, the method of using the dental implant drill according to the invention reduces the number of drill bits during a surgery from multiple implant drill bits in series (about 3-6) to the use of only one drill bit.

Using the internally irrigated dental implant drill 100 reduces the time and number of drill bits during the osteotomy procedure, which allows for a minimally invasive osteotomy, leading to maximum stability of the implant at placement, while at the same time not risking bone burning and necrosis.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above.

What is claimed:

1. A dental implant drill bit, the dental implant drill bit comprising;
a longitudinal body portion having guide markings;
a central bore longitudinally extending through the longitudinal body portion;
a proximal end portion having a drill tip;
a flange portion disposed in a medial portion of the longitudinal body portion; and
a connecting portion having a distal end portion, wherein the distal end portion is configured to couple to a driver.

2. The drill bit of claim 1, wherein the central bore is configured to provide irrigation.

3. The drill bit of claim 1, wherein the drill bit is disposable.

4. A dental implant drill, the dental implant drill comprising:
a drill bit, the drill bit comprising a longitudinal body portion having guide markings;
a central bore longitudinally extending through the longitudinal body portion; a proximal end portion having a drill tip; a flange portion disposed in a medial portion of the longitudinal body portion; and a connecting portion having a distal end portion; and
a driver, wherein the distal end portion is configured to couple to the driver.

5. The dental implant drill of claim 1, wherein the central bore is configured to provide irrigation.

6. The dental implant drill of claim 1, wherein the drill bit is disposable.

7. A method of using a dental implant drill for a dental osteotomy comprising:
drilling a hole using the dental implant drill, wherein said hole is of a desired width and depth for receiving an implant, and wherein the dental implant drill comprises a drill bit, the drill bit comprising a longitudinal body portion having guide markings, a central bore longitudinally extending through the longitudinal body portion, a proximal end portion having a drill tip, a flange portion disposed in a medial portion of the longitudinal body portion, and a connecting portion having a distal end portion, wherein the distal end portion is configured to couple to a driver; and
internally irrigating the drill bit through the central bore while drilling said hole.

8. The method of claim 7, wherein the drill bit for drilling the hole for receiving the implant is singular.

9. The method of claim 7, wherein the drill bit is disposable.

10. A dental implant drill bit kit comprising one or more drill bits, wherein each of said one or more drill bits comprises:
a longitudinal body portion having guide markings; a central bore longitudinally extending through the longitudinal body portion; a proximal end portion having a drill tip; a flange portion disposed in a medial portion of the longitudinal body portion; and a connecting portion having a distal end portion, wherein the distal end portion is configured to couple to a driver.

11. The dental implant drill of claim 10, wherein the central bore is configured to provide irrigation.

12. The dental implant drill bit kit of claim 10, wherein the one or more drill bits are of different widths.

13. The dental implant drill bit kit of claim 10, wherein the flange portion of each of the one or more drill bits is of a different depth.

14. The dental implant drill bit kit of claim 10, wherein the one or more drill bits are disposable.
